(19) United States
(12) Patent Application Publication
(10) Pub. No.: US 2023/0028833 A1
(43) Pub. Date: Jan. 26, 2023

(54) Machine Learning Based Strength Training System and Apparatus Providing Technique Feedback

(71) Applicants: Natalie NG, Boston, MA (US); Matthew KATZMAN, Boston, MA (US)

(72) Inventors: Natalie NG, Boston, MA (US); Matthew KATZMAN, Boston, MA (US)

(21) Appl. No.: 17/382,558

(22) Filed: Jul. 22, 2021

(51) Int. Cl. A63B 71/06; G06N 20/00; G06K 9/00

(52) U.S. Cl. CPC A63B 71/0622; G06K 9/00342; G06N 20/00

(57) ABSTRACT

An exercise form analysis and feedback system (EFAF) including at least one sensor, at least one local movement data receiver, an analysis and feedback processing unit (AFPU) and a feedback display. The EFAF system of the present invention obtains lift movement data through the one or more sensors as lift movements are performed. This lift movement data may, in turn, be transmitted to one or more local movement data receivers such that the AFPU may operate on the lift movement data to provide real-time or near real-time form/technique feedback to the user via a feedback display. The system of the presentation invention uses machine learning techniques to provide feedback on lift quality aspects based on data associated with previous lifts and external data as applicable.

### CROSS-REFERENCE TO RELATED APPLICATIONS

Not applicable.

### FIELD OF THE DISCLOSURE

Disclosed embodiments relate to fitness and exercise equipment and the use thereof, and more specifically, to devices and techniques for providing feedback to users of weightlifting equipment regarding their form and technique in using such equipment.

### BACKGROUND

It is well known that exercise and physical activity are important aspects in promoting good health and longevity. There are a practically unlimited number of ways for people to engage in such activities depending on the activities they enjoy and/or the types of results they are trying to achieve.

One popular set of exercises is weightlifting. Participants in weightlifting activities are typically looking to increase strength, tone their physical appearance and/or increase physical stamina. Weightlifting activities can take many forms and may include the use of barbells, dumbbells, and various strength training machines which have integrated sets of weights all of which require the participant to use muscles to overcome the gravitational force that the weights provide in connection with accomplishing the applicable motion associated with the particular exercise.

While other types of exercises such as those typically associated with cardio based activities (e.g. treadmills, elliptical trainers, stationary bicycles, etc.) require minimal training or experience in terms of the correct form to use, weightlifting exercises do typically require that a participant have at least some experience with and/or understanding of proper form associated with the particular lift. Failure to undertake these weightlifting exercises using proper form and technique can lead to various undesirable results. For example, improper form can result in less than desirable training results such as when the muscles targeted are not the ones primarily worked because the lift is not properly performed. Worse yet, improper form or technique can lead to injury including those that cause long term damage to bone and/or muscle structure which may result from performing one or more lifts improperly over a period of time.

One solution available to those who desire to engage in weightlifting but do not have the specific experience to facilitate good form and technique in performing lifts is to engage an experienced trainer to observe and provide feedback on the participant’s workouts. Unfortunately, however, good trainers can be hard to find and/or expensive since they typically charge an hourly rate for each workout for which they provide training. Even still, some “experienced” trainers may not actually know the proper form for certain exercises or they may not be able to tailor their understanding of the proper form to the various different body types of their clients.

Thus, it would be desirable to provide a technological solution to monitoring weightlifting exercises when undertaken by a participant through which the participant could receive feedback on whether they are executing the various exercises using the proper form and technique. While there exist some products and solutions that are intended to meet this need, they do suffer from various drawbacks.

In the context of solutions associated with the use of “free weights” (i.e. barbells and dumbbells with the ability to load various weight loads on the bar), there exist solutions wherein sensors are affixed to the bars such that they provide data on the movement of a barbell during the lifting motion. There are also other solutions that involve scanning with cameras and/or the laser optics to assess the motion of lifts with the goal of providing feedback to the activity participant. In addition, there exist solutions in which sensors are placed on the body of the participant in order to assess body movement during the performance of a lift.

While the foregoing solutions are helpful, they do not address a number of desirable aspects which are provided by the present invention. For one, in many cases, the available sensors and deployment thereof do not provide the full amount of data necessary to make a good determination as to form. Additionally, existing systems do not account for deviations in proper form based on differences in body types such as height and weight differences as well as the particular exercise being performed by the participant.

In addition, existing solutions for monitoring and providing feedback in connection with weightlifting movements do not generally provide the ability for such systems to improve the quality of their feedback over time or in connection with movement data obtained from the participant’s previous lifts or even with data obtained from lifts performed by other users of the solution.

### SUMMARY

It is to be understood that both the following summary and the detailed description are exemplary and explanatory and are intended to provide further explanation of the present embodiments as claimed. Neither the summary nor the description that follows is intended to define or limit the scope of the present embodiments to the particular features mentioned in the summary or in the description. Rather, the scope of the present embodiments is defined by the appended claims.

It is a primary object of the present invention to provide a system and methodology through which weightlifting activity participants can receive feedback regarding the form that they demonstrate while undertaking one or more weightlifting activities.

It is another object of the present invention to provide a system and methodology in which one or more sensors are provided for detecting and transmitting data associated with one or more metrics resulting from the movement of a weightlifting element such as a barbell or a dumbbell during a lifting exercise.

It is a still further object of the present invention to utilize such movement data to analyze and provide feedback to a user in respect of the form and technique associated with various weightlifting exercises undertaken by the user.

It is an even further object of the present invention to provide feedback with respect to various form aspects associated with a lift such as physical positioning, depth, balance, acceleration and the like.

It is a still further object of the present invention to implement various machine learning techniques to improve the quality and quantity of feedback which may be provided to the user as the user and/or other users perform lifting exercises over time.

It is a yet further object of the present invention to provide novel sensor constructs such as a knee sleeve which generates data indicative of form associated with various lifting exercises.

An aspect of the invention, in one embodiment thereof, includes an exercise form analysis and feedback system (EFAF) including at least one sensor, at least one local movement data receiver, an analysis and feedback processing unit (AFPU) and a feedback display. The EFAF system of the present invention obtains lift movement data through the one or more sensors as lift movements are performed. This lift movement data may, in turn, be transmitted to one or more local movement data receivers such that the AFPU may operate on the lift movement data to provide real-time or near real-time form/technique feedback to the user via the feedback display.

In some embodiments, machine learning techniques may be applied so as to improve the feedback provided to the user. These techniques may include the use of lift movement data previously obtained from the user to further refine the feedback provided by the EFAF of the present invention. In other embodiments, lift movement data from other users as well as the analysis thereof, may be used to refine the feedback provided by the EFAF according to various aspects of the present invention. In preferred embodiments of the present invention, unique aspects of each user are taken into account in connection with the analysis and feedback provided. These aspects may include body characteristics of the user such as height, weight, limb lengths and other unique aspects of the user performing the lift exercises.

According to some embodiments of the present invention, various novel sensor constructs may be used to obtain lift movement data which is useful in providing accurate form feedback. One such construct is a knee sleeve which fits over the user’s knee and incorporates various sensors which detect knee movements in order to assess various “quality of lift” metrics such as knee buckling, squat depth and other characteristics associated with providing feedback in connection with weightlifting activities.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. Embodiments herein will be more particularly described in conjunction with the following drawings wherein:

FIG. 1 is an illustration of the EFAF system of the present invention in a preferred embodiment thereof, providing exercise form and technique feedback to a user in accordance with the teachings of the present invention;

FIG. 2 is an illustration of an embodiment of the data processing workflow applied to raw sensor data in connection with the EFAF of the present invention in a preferred embodiment thereof;

FIG. 3 is an illustration of a barbell including sleeve sensors used for capturing raw movement data according to the teachings of the present invention in one embodiment thereof;

FIG. 4 is a more detailed illustration of a barbell sleeve sensor according to the teachings of the present invention;

FIG. 5 is an illustration showing the placement of a knee sleeve with sensors on a user’s knee according to one preferred embodiment of the present invention;

FIG. 6 is a more detailed illustration of a knee sleeve and its component sensors according to the teachings of the present invention;

FIG. 7 is a flowchart showing the primary steps in connection with obtaining and processing sensor data and providing feedback to the user regarding weightlifting form and technique according to the teachings of the present invention in a preferred embodiment thereof;

FIG. 8 is an illustration of exemplary raw and transformed data associated with a series of lifts and illustrates the application of such data when the system is in the manual learning mode in accordance with the teachings of the present invention;

FIG. 9 is an illustration of exemplary raw and transformed data associated with a series of lifts and illustrates the application of such data when the system is not in the manual learning mode in accordance with the teachings of the present invention; and

FIG. 10 is an illustration demonstrating data capture and the use of that data to make and report lift quality determinations in accordance with the teachings of the present invention.

### DETAILED DESCRIPTION

The present disclosure will now be provided in terms of various exemplary embodiments. This specification discloses one or more embodiments that incorporate features of the present embodiments. The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. The skilled artisan will appreciate that a particular feature, structure, or characteristic described in connection with one embodiment is not necessarily limited to that embodiment but typically has relevance and applicability to one or more other embodiments.

In the several figures, like reference numerals may be used for like elements having like functions even in different drawings. The embodiments described, and their detailed construction and elements, are merely provided to assist in a comprehensive understanding of the present embodiments. Thus, it is apparent that the present embodiments can be carried out in a variety of ways, and does not require any of the specific features described herein. Also, well-known functions or constructions are not described in detail since they would obscure the present embodiments with unnecessary detail.

The description is not to be taken in a limiting sense, but is made merely for the purpose of illustrating the general principles of the present embodiments, since the scope of the present embodiments are best defined by the appended claims.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase "and/or," as used herein in the specification and in the claims, should be understood to mean "either or both" of the elements so conjoined, i.e., elements that are conjunctively present in some cases and disjunctively present in other cases. Multiple elements listed with "and/or" should be construed in the same fashion, i.e., “one or more” of the elements so conjoined. Other elements may optionally be present other than the elements specifically identified by the "and/or" clause, whether related or unrelated to those elements specifically identified. Thus, as a non-limiting example, a reference to "A and/or B", when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of,” or “exactly one of,” or, when used in the claims, “consisting of,” will refer to the inclusion of exactly one element of a number or list of elements. In general, the term “or” as used herein shall only be interpreted as indicating exclusive alternatives (i.e. "one or the other but not both") when preceded by terms of exclusivity, such as "either," "one of," "only one of," or "exactly one of.” "Consisting essentially of," when used in the claims, shall have its ordinary meaning as used in the field of patent law.

As used herein in the specification and in the claims, the phrase "at least one," in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as "comprising," "including," "carrying," "having," "containing," "involving," "holding," "composed of," and the like are to be understood to be open-ended, i.e., to mean including but not limited to. Only the transitional phrases “consisting of” and “consisting essentially of” shall be closed or semi-closed transitional phrases, respectively, as set forth in the U.S. Pat. Office Manual of Patent Examining Procedures, Section 2111.03.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments. As used herein, the term “and/or” includes any and all combinations of one or more of the associated listed items. As used herein, the singular forms "a", "an" and "the" are intended to include the plural forms as well, unless the context clearly indicates otherwise.

The word “exemplary” is used herein to mean “serving as an example, instance, or illustration.” Any embodiment described herein as “exemplary” is not necessarily to be construed as preferred or advantageous over other embodiments. Additionally, all embodiments described herein should be considered exemplary unless otherwise stated.

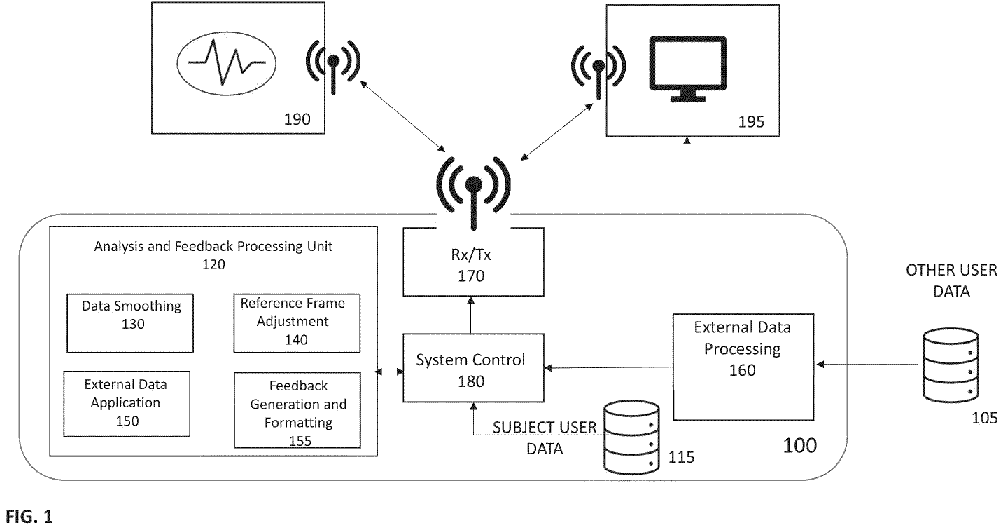

With reference now to FIG. 1, a description of the EFAF system 100 of the present invention and the operation thereof is provided in one embodiment thereof. EFAF system 100 includes multiple components which allow for the receipt of raw sensor data, processing functionality to act upon the raw sensor data and reporting functionality to provide feedback data which such data, in some embodiments, may be formatted such that it is displayed on a display device for a user to view.

In preferred embodiments of the present invention, EFAF system 100 receives raw sensor data which is collected by one or more sensors 190. Sensors 190 may be located in various locations associated with weightlifting activities and may be of many different forms such that sensors 190 are able to collect the raw data required to make determinations regarding a user's form and/or technique in performing various lifts. For example, and as more fully described below, sensors 190 may be implemented as sleeves located on a barbell. Alternatively, sensors 190 may be located within a knee sleeve which is placed on a user’s knee in order to determine various movements in the area of a human knee. Many other sensor configurations are possible while remaining within the scope and spirit of the present invention.

In preferred embodiments of the present invention, sensors 190 communicate wirelessly via known communication protocols with EFAF system 100. Alternatively, the communication may occur via a wired connection. In any case, EFAF 100 preferably includes Rx/Tx component 170 which is configured to receive and transmit data either wirelessly or via a wired connection. Communication protocols may include, for example, short range protocols such as Bluetooth and/or WiFi connections.

In accordance with the teachings of the present invention, sensors 190 collect “raw” movement data such as the acceleration of the barbell in three different dimensions and gyroscopic data as more fully described below. In addition, other data such as knee bend data may be obtained via sensors 190 located within a knee sleeve in accordance with the teachings of the present invention and as more fully described below. This raw data is transmitted from sensors 190 to EFAF system 100 via a transmitter associated with sensor 190 and Rx/Tx component 170 within EFAF system 100.

In some embodiments of the present invention, EFAF system 100 may be implemented as a personal computer or other computing device with software specifically configured to carry out the operations described herein. EFAF system 100 is under the control of system control 180 which directs the sequence of operations undertaken by EFAF system 100. In some embodiments system control 180 may be a central processing unit of a personal computer as directed by the specially configured software referenced above.

EFAF system 100 also includes analysis and feedback processing unit (AFPU)120 which itself preferable includes multiple sub-components. One such component is data smoothing module 130 which takes the raw movement data received by EFAF system 100 and applies one or more filters (as more fully described below) to smooth the data such that it is usable in making form and technique analysis determinations and recommendations as discussed herein. In addition, EFAF system 100 may also include reference frame adjustment module 140. This module applies known data regarding the body type and measurements of a user so that the form and technique analysis can take into account such factors in implementing the analysis. This is more fully discussed below.

AFPU 120 may also include external data application module 150. This module functions to employ external data regarding users other than the subject user in performing form and technique analysis. This external data is obtained via various sources of such external data denoted as “Other User Data” 105 shown in FIG. 1. Once this Other User Data is received, it may be processed and/or formatted for use in connection with the processing undertaken by AFPU 120 by external data processing module 160.

A key aspect of the present invention is to employ machine learning techniques to enhance form and technique analysis for each user over time. This is accomplished and implemented by AFPU using “Subject User Data” which is collected and stored in the applicable database 115 under the control of system control 180 and AFPU 120.

In particular, as users perform lifts and more and more sensor data is obtained, EFAF 100 may use this “subject user data” to enhance the form and technique analysis results associated with a particular user. In addition, and as mentioned above, “other user data” may also be employed to enhance the analysis results as more fully described below. In addition to “subject user data” and “other user data”, the reference frame data described above is also preferably used by EFAF 100 to optimize the analysis. Finally, external human input may be accepted by EFAF 100 and used in optimizing the analysis. In this case, human input may include correlation data representing, for example, the correlation of raw data and/or smoothed data to “good lifts” and/or “bad lifts” indicative of which data is indicative of good form and which is not.

In some embodiments, and as more fully described below, human users such as trainers can provide initial correlation data to EFAF 100 representing which type of raw data and/or smoothed data correlates to “good” lifts and which correlates to “bad” lifts. In addition, this correlation data may include information indicative of particular, specific errors in form based on the presence and/or absence of specific raw data and/or actionable data values obtained from sensors 190 or derived through system processing as lifts are performed.

Accordingly, machine learning techniques may then be applied as additional lift data is obtained from subject users and other users and possibly with additional human input such that correlations between raw/smoothed data obtained from lifts and determinations as to form and technique may be optimized. In preferred embodiments, the machine learning database is updated only in connection with manual learning mode where human input is provided in connection with actionable data. However, in other embodiments, the machine learning database may be updated solely based on additional actionable data as it is generated by the system when lifts are performed.

Returning to FIG. 1 and the specific components of EFAF 100, AFPU 120 preferably also includes feedback generation and formatting module 155. This module functions to take the determinations made regarding form and technique for lifts and format them so that a user may view them on display 195. Alternatively, or in addition, EFAF 100 may provide auditory feedback to the user regarding lift quality. Data associated with the results may be wirelessly transmitted by EFAF 100 to display 195 or a wired connection may be used. In preferred embodiments, a user may interact with EFAF 100 to customize the formatting and/or content of the analysis results as displayed on display 195. Similarly, a user may interact with EFAF 100 to initiate and configure EFAF 100 generally as well as to initiate the process under which lifts are undertaken and sensors 190 configured and utilized to obtain and provide raw data to EFAF100. Interaction between the user and EFAF system 100 may be accomplished via a keyboard and/or mouse or other input device which communicates with EFAF 100.

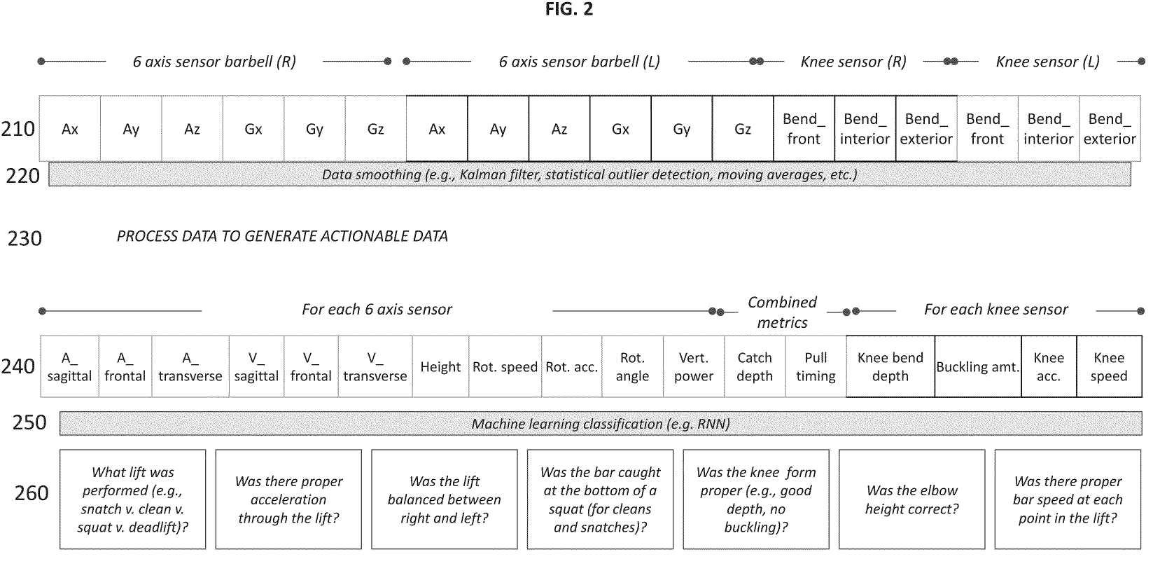

Turning now to FIG. 2, an exemplary workflow for receiving and processing raw data obtained from sensors 190 in order to make form and technique determinations is now discussed. With reference to FIG. 2, row 210 represents exemplary raw sensor data that may be obtained via sensors 190. In this example, one six axis barbell sleeve sensor is located on each of the right and left ends of a barbell and a knee sleeve with three bend sensors is located on each one of the user’s knees. As will be readily apparent to one of skill in the art, many different/alternative sensor configurations can be used while still remaining within the scope and spirit of the present invention.

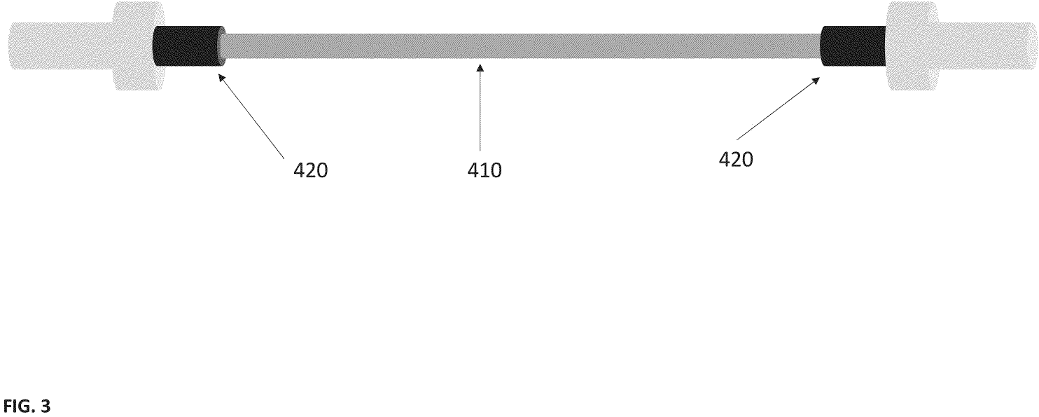

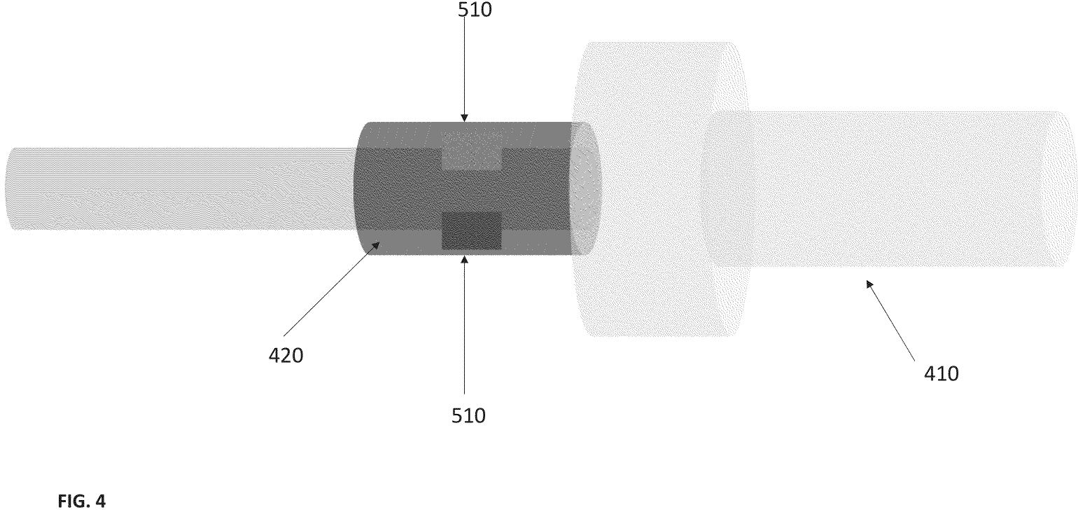

In this case, and with reference to FIGS. 3-6, barbell sleeve sensors 420 are located at each end of barbell 410. In preferred embodiment, these sleeve sensors 420 are constructed so that they may be attached and detached from barbell 410 using, for example, a Velcro type fastener arrangement. Preferably, sleeve sensors are located as close to the distal ends of barbell 410 where the weights are but without interfering with the placement of the weights themselves. In preferred embodiments sleeve sensors are located inside the barbell collars to prevent undesirable rotation of the sensors. With reference specifically to FIG. 4, sleeve sensor 420 may contain one or more six-axis sensors 510 embedded within the sleeve. Examples of sensors that may be used for this purpose include the Adafruit LIS3DH Triple Axis Accelerometer model ADA2809 available from Adafruit .

In preferred embodiments, six-axis sensors 510 obtain three different acceleration measurements comprising one in each of the x, y and z dimensions. In addition, six-axis sensors 510 further obtain three different gyroscopic measurements comprising rotation about each of the x, y and z axes. As will be understood by one of skill in the art, these gyroscopic rotation axes are equivalent to the roll, pitch and yaw rotational movements of an aircraft. The three acceleration measurements are represented in row 210 as Ax, Ay and Az and the three gyroscopic measurements are represented as Gx, Gy and Gz. These six measurements are provided for each of the left and right sides of barbell 510 comprising a total of twelve measurements.

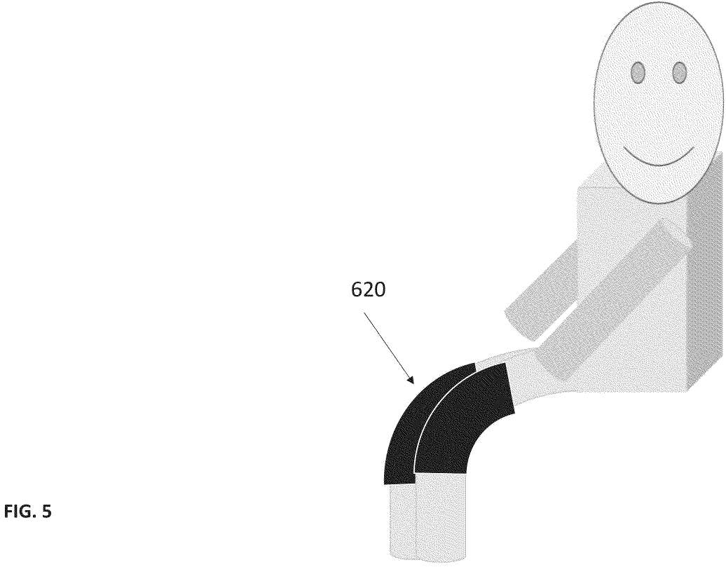

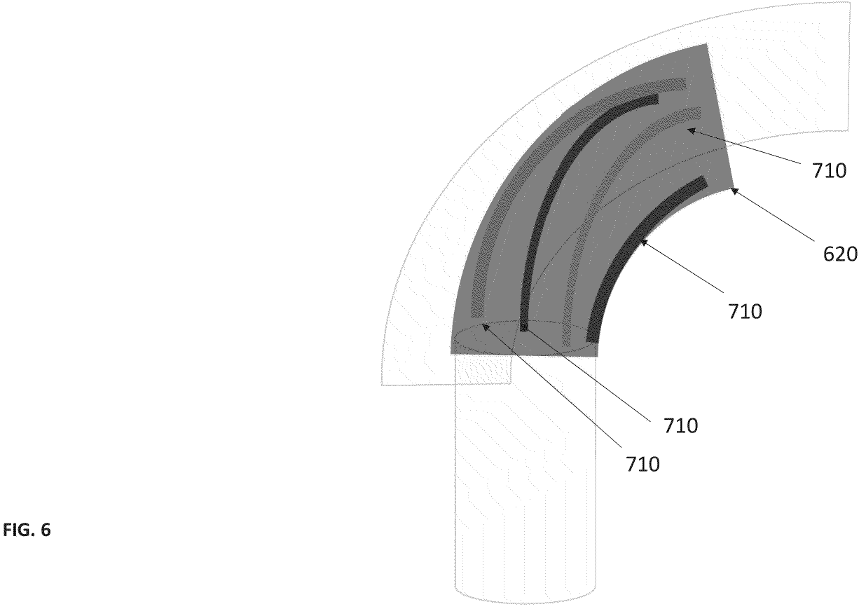

In addition to barbell sensor sleeves 420, and with reference to FIGS. 5 and 6, EFAF 100 may also employ other sensor configurations such as knee sleeve 620 which may be placed over one or both of a user’s knees. Knee sleeve 620 may comprise a flexible fabric sleeve which either slips over the user’s foot and which is raised up to the knee or, alternatively, knee sleeve 620 may be opened and closed into a tube shape via a Velcro type fastener arrangement after being located in the user’s knee area.

Knee sleeve 620 may include any number of bend sensors 710 which may comprise fabric strips embedding sensor components. While FIG. 6 shows four such bend sensors 710, more or less sensors are possible while remaining within the scope and spirit of the present invention. In any event, the purpose of knee sleeve 620 and bend sensors 710 is to obtain data representative of the amount of knee bend a user undertakes in connection with weightlifting movements. Bend sensors 710 measure resistance and are placed in varied locations within knee sleeve 620 in order to detect depth and direction of compression as weightlifting movements are performed. In preferred embodiments of the present invention, examples of bend sensors 710 which may be employed include the Adafruit Short Flex Sensor model ADA1 070 available from Adafruit (www.adafruit.com).

Raw data collected by sensors 190 is provided to EFAF 100. Raw data captured during the lift exercise is captured in time series fashion during the time frame that the complete lift exercise is undertaken. In some embodiments, raw data is captured every 20 ms although many other time series intervals may be used without departing from the scope or spirit of the present invention. Actual time series intervals used will depend to some extent on storage capacities and processing capabilities.

In preferred embodiments, captured raw data is processed by a filter (see row 220) under the control of data smoothing component 130. In some embodiments, data smoothing may be performed using a Kalman filter although other suitable filters may be used. The purpose of data smoothing is to eliminate noise and statistical inaccuracies associated with the collective raw sensor data obtained by sensors 190 and provided to EFAF 100. The data smoothing process is applied to the time series data obtained by sensors 190 over time in respect of the series of data obtained in connection with an exercise lift movement.

In preferred embodiments, data smoothing comprises interpolation of data that is identified as outlier data. This interpolation seeks to fill in data that would be expected to occur in the time series between the nearest data captures both before and after the outlier data. Alternatively, in some embodiments, data smoothing occurring in row 220 may comprise eliminating data that is clearly an outlier and treating it as it were never captured and eliminating a data point at the time interval associated with the outlier data.

In preferred embodiments, the smoothed data is then processed by AFPU 120 to generate actionable data (row 230) for each time point of data captured via sensors 190. With respect to the six axis barbell sensors, for example, the following actionable data may be generated as follows:
The change in gyroscopic rotation (Gz) is integrated to reclassify “down” relative to the user’s reference frame in association with reference frame adjustment component 140. User’s reference frame may be input by the user and provided to EFAF 100. For example, the user may be prompted to enter their height and weight. Other user reference data may also be provided by the user (e.g. user gender, user age, bar weight, etc.) and such data may be used in the calculation.
The accelerometer data is transformed to acceleration measurements in each dimension (x, y, z) relative to the user's reference frame; and
Differences between the right and left sensors, including, for example, acceleration in the y dimension, as barbell 410 is moved through a lift, are identified to determine issues such as unbalanced movement.

In preferred embodiments, and with respect to knee sleeve 620 raw data, for each time point associated with the captured raw data, the following actionable data may be calculated, for example:
Absolute squat depth may be measured if the user is performing a squat type exercise
Speed of squat movement may also be determined using a first derivative of the acceleration measurement obtained in one or more dimensions.
Knee buckling may be detected and reported via the available knee sleeve 620 data

Exemplary actionable data available in preferred embodiments of the present invention based on the processing occurring in row 230 of FIG. 2 is shown in row 240 of FIG. 2. This set of actionable data is merely an example and other data relating to lift activities could be added and/or substituted while still remaining within the scope and spirit of the present invention. Exemplary calculations to obtain the actionable data based on the smoothed raw data is provided in the following table:

| | |
|---|---|
| Actionable Data Element | Description of Calculation |
| A_sagittal | acceleration along three axes in the lifter’s reference frame Calculated by integrating gyroscopic rotation to determine lifter’s reference frame relative to the sensor's reference frame, and transforming the sensor’s reference frame to the lifter’s reference frame via multiplication with rotation matrix. |
| A frontal | |
| A transverse | |
| V_sagittal | Calculated by integrating acceleration over time in each direction (above three metrics) |
| V_frontal | |
| V transverse | |
| Rotational acceleration | Represents rotation along the axis of the bar (Gx); can also be calculated for the two other axes (Gy and Gz) |
| Rotational speed | Calculated by integrating rotational acceleration over time |
| Rotational angle | Calculated by integrating rotational speed over time |
| Vertical power | Requires weight of bar as input; calculated as mass * gravity * V_sagittal |
| Catch depth | Knee bend depth (below) at time of minimal bar height during the lift |
| Pull Timing | V_sagittal calculated solely between start of lift and next knee bend |
| Knee bend depth | Maximum bend of front bend sensor of knee during lift |
| Knee bend buckling | Maximum bend of interior bend sensor during lift |
| Knee speed | Rate of change in front bend sensor |
| Knee acceleration | Numeric differential of knee speed |

Next, row 250 in FIG. 2 illustrates the machine learning capabilities of the present invention. In particular, certain questions regarding lift quality may be posed and related answers to those questions may be provided using the actionable data generated above. Row 260 in FIG. 2 shows examples of such questions. Descriptions of those questions is provided in the table below:

| | |
|---|---|
| Question | Description of question |
| What lift was performed? | Decides what lift was being performed based on the features present in the readings - in this case we assume a clean. |
| Proper acceleration? | Was the initial pull forceful enough, or too weak? Was the acceleration almost entirely in the sagittal direction, or was there significant transverse force? |
| Lift balanced? | Was any frontal force or anterior-posterior rotation detected? |
| Catch at bottom? | Was the knee bend sufficient at the time of minimal bar height during the lift? |
| Knee form proper? | How similar were the interior and exterior bend sensor readings to each other? Were (R) and (L) knees similar? |
| Elbow height correct? | Was the mediolateral rotation sufficient to ensure wrists and elbows are at a proper height? |
| Proper bar speed? | Was V_sagittal low at any surprising point during the lift? |

Again, it will be readily understood by one of ordinary skill in the art that the above questions are merely examples and various other questions could be posed and answered while still remaining within the scope of the present invention.

As noted above, the questions may be answered based on pairing the actionable data obtained with respect to the current lift with available machine learning data as lifts by this user and other users are conducted. In the manual learning mode, an experienced weightlifter or instructor could input answers to the questions for the lift that just occurred (e.g. there was proper acceleration but the knee form was improper) and those results will be matched up with the actionable data generated from the lift to populate the machine learning database with intelligence tying the question answers to applicable actionable data. For example, if a lifter performs a lift that is deficient in a number of ways (e.g. knee form bad, elbow height incorrect, improper bar speed etc.), the actionable data generated from that bad lift in time series form will closely match to actionable data contained in the machine learning database indicative of such bad lift characteristics.

In a manual learning mode, a coach/trainer/experienced lifter can assist with “training” the system by manually answering questions associated with lifts after they occur. Once this data has been captured, the system will be better at accurately answering the questions posed. Outside of manual learning mode, as users perform lifts, the actionable data can be matched against the question answers and the decisioning as to how various questions are answered can be refined over time as more lift data becomes available.

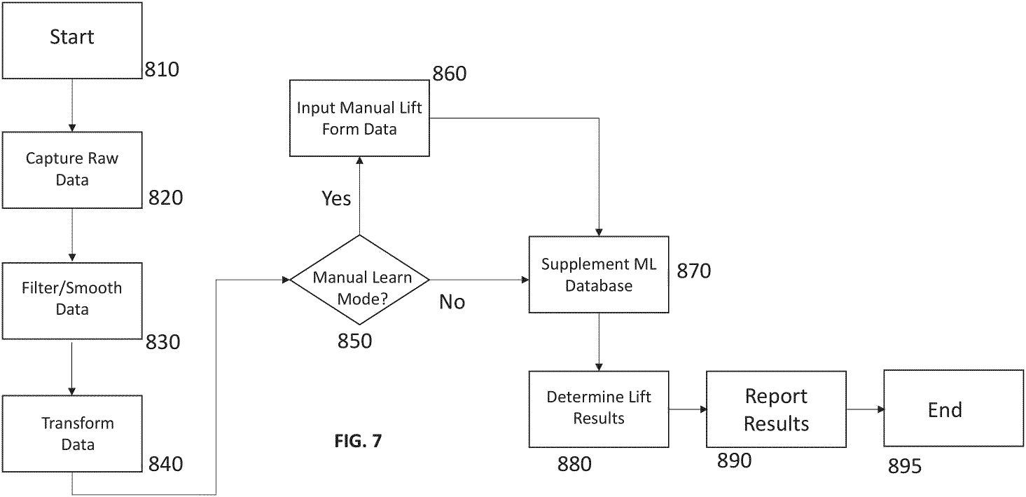

FIG. 7 is a flowchart representing steps, in a preferred embodiment, through which EFAF obtains sensor data, processes it and applies machine learning techniques to provide optimized lift quality feedback to a user undertaking a weightlifting exercise. A description of that process, in connection with the FIG. 7 is now provided. The process begins at step 810. At step 820 raw data is captured by one or more sensors 190 as described above. At step 830, the raw data is filtered and smoothed such that outlier data is either discarded or interpolation is undertaken to address any errant data captured by one or more sensors 190 during the lift activity. At step 840, the filtered/smoothed data is processed as described above to generate actionable data which can be used to make determinations regarding lift quality. As described above, and as applicable, this processing may include taking integrals or derivatives of the data or applying other data transformations as needed to generate data such as actual bar acceleration, rotational speed, velocities and the like.

Next, at step 850, a determination is made as to whether the EFAF system 100 is in a learning mode and still requires additional manual input or not. If not in manual learn mode, processing proceeds to step 870 which is described below. Alternatively, if the system is in manual learn mode, processing proceeds to step 860 wherein a user such as a coach or experienced weightlifter enters information regarding the lifts he or she just did into EFAF system 100. As described above and for example, the user may enter various answers to questions regarding the lift that the user just did such as whether the knees where buckled, whether the elbows were bent the correct amount and other question/answer pairs. This input is paired with the lift data captured by EFAF system 100. Processing then continues to step 870.

At step 870 the machine learning database is supplemented with the new lift data associated with the lift just performed. This data is stored in the database to achieve improved results via the machine learning techniques described above. As described above, if EFAF system 100 is in manual learn mode, the data stored also includes additional data which guides the determination of lift quality associated with the lift data generated and stored.

At step 880, the most up to date data regarding the mapping of lift data to actionable lift quality results is used to generate lift results. Based on the data associated with the lift just performed, lift results, as described above, may provide answers to specific questions regarding lift quality such as whether elbow bend was correct, whether there was proper bar speed, whether knee form was correct and the like. At step 890, these results may be reported to the user such as through a user interface available on display 195. The process then ends at step 895.

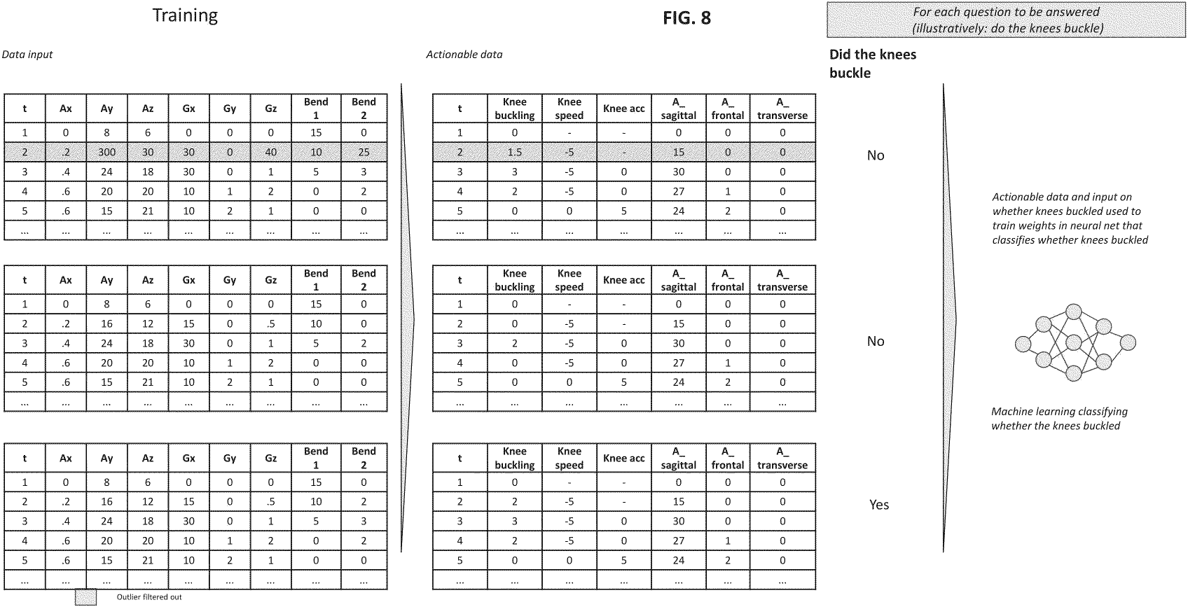

Now, with reference to FIG. 8 and in furtherance of the description of the system and methodology of the present invention herein, an example of how lift quality feedback in connection with a user performing a squat is determined using exemplary raw sensor data. In the case of FIG. 8, EFAF system 100 is in “manual learn mode” such that following the lifting exercise, a user will input answers to the question of whether the knees buckled manually for use by the system in connection with future lifts by this user and/or other users.

As can be seen in FIG. 8, the three tables on the left represent raw data captured in time series (e.g. at times 1-5) for each of three successive lifting motions (e.g. three reps of a squat exercise). The raw data captured at each time point includes acceleration in three dimensions (Ax, Ay, Az), gyroscopic/acceleration measurements (Gx, Gy, Gz) and two different knee bend values.

The first item of note is that system 100 operates using the smoothing filter to interpolate data for time interval t2 since the data captured is clearly an outlier. Further, it can be seen that the raw data is processed to generate the following actionable data associated with each time interval across the three lift repetitions: knee buckling value, knee speed value, knee acceleration value and three different acceleration values (sagittal, frontal and transverse). These values are determined according to the calculations described above starting with the captured raw data.

In the manual learning mode (training mode), the user then enters what he or she actually viewed during the lift exercise. In this case, the user enters that the knees did not actually buckle during either the first or second repetitions but that they did during the third repetition. The actionable data and the answer to the question as to whether the knees buckled during each repetition is stored within the machine learning database for use in answering this question more accurately for future lifts performed by this and/or other lifters. As noted previously, personal data such as the lifter’s age, gender, height and weight may also be tied to the data record included in the machine learning database.

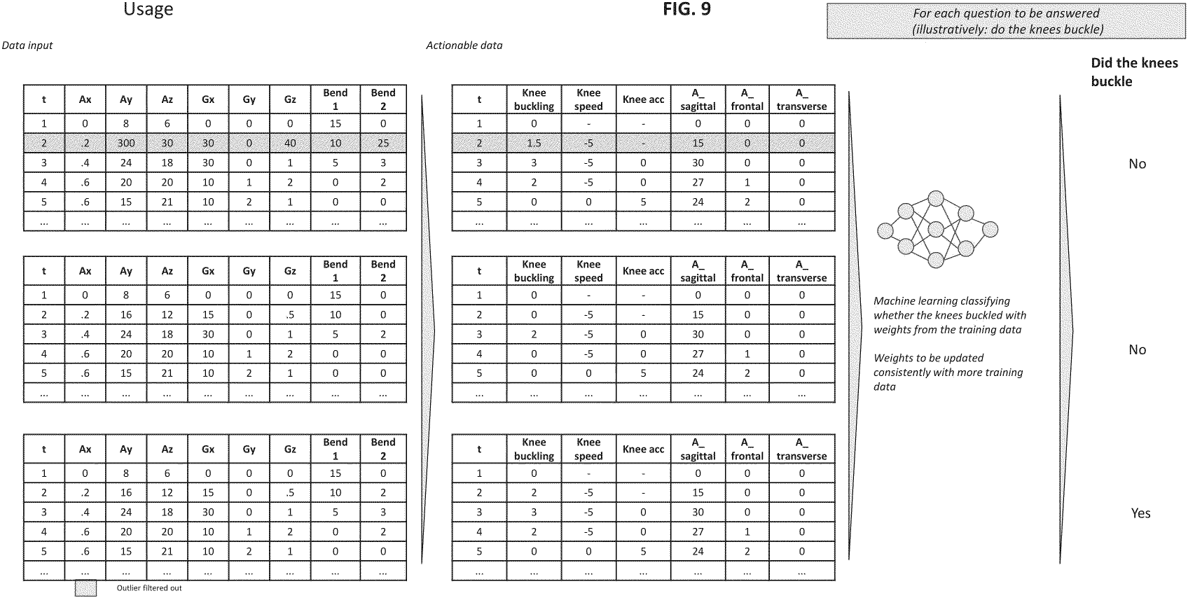

Now with reference to FIG. 9, data capture when not in the “manual learn mode” is presented and discussed. In this case, again the second time interval in the first repetition requires smoothing and the interpolated data is included in the actionable data table. Further, the actionable data is matched against the data in the machine learning database from the previous lifts (e.g. the lifts done in connection with FIG. 8) and the system is able to answer the question as to whether the knees buckled during the lifts consistent with previous data/results matching.

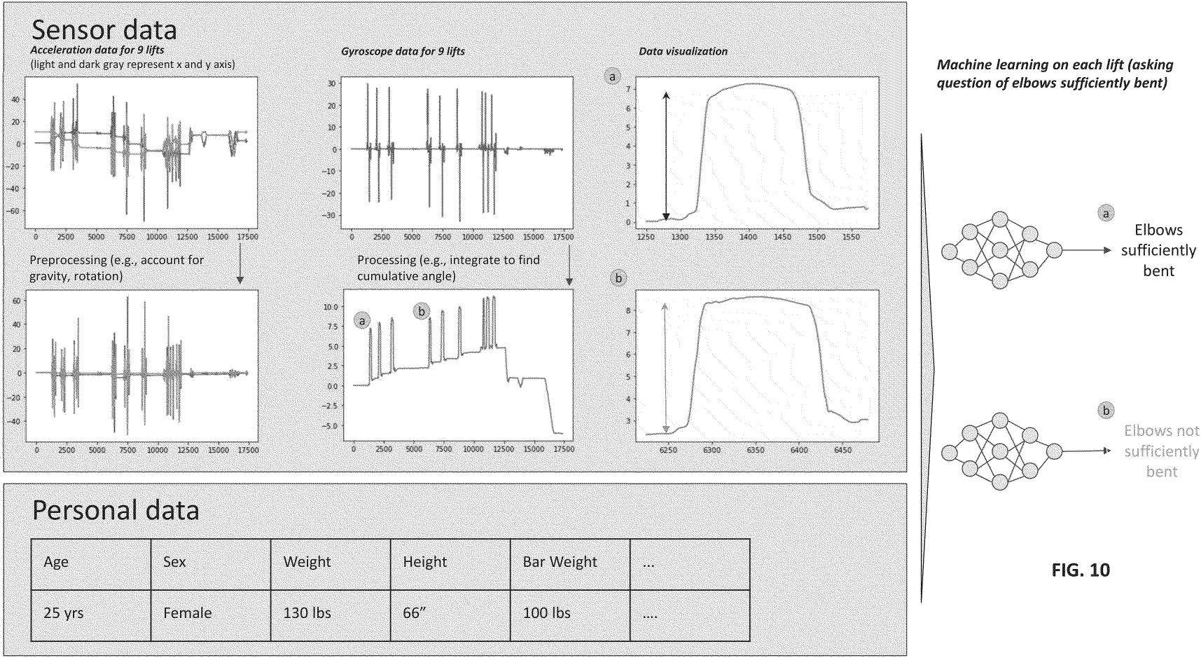

FIG. 10 is an illustration presenting results involving data capture and the visualization of that data in connection with a determination by the system of the present invention as to whether the lifter’s elbow’s were sufficiently bent during the lifting exercise. As can be seen, the raw data at the top left column and top middle column (acceleration data and gyroscope data, respectively) of the figure is processed by the system to generate actionable data as shown in the graphs below each respective raw data graph. As discussed above, the bottom graphs represent the processing of the time series data for each of the nine repetitions in this example to generate the actionable data.

The resulting elbow angle graph in the middle column thus shows the processed elbow angle data for each of the nine repetitions. In the case of the repetition labeled as “a” with the spike in the graph enlarged in the right most column, it is determined, based on the delta between one extreme of the elbow angle at one time vs. the other extreme of elbow angle at a later time, that the elbow was sufficiently bent during that repetition. By contrast, for repetition “b”, the delta in elbow angle difference is less than ideal and thus the system will report that the elbow was not sufficiently bent during this repetition as may be detected during, for example a “clean” type of lift exercise.

The present invention is not limited to the particular embodiments illustrated in the drawings and described above in detail. Those skilled in the art will recognize that other arrangements could be devised. The present embodiments encompass every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the present embodiments have been described with reference to specific illustrative embodiments, modifications and variations of the present embodiments may be constructed without departing from the spirit and scope of the present embodiments as set forth in the following claims.

Although the present embodiments have been described in detail, those skilled in the art will understand that various changes, substitutions, variations, enhancements, nuances, gradations, lesser forms, alterations, revisions, improvements and knock-offs of the embodiments disclosed herein may be made without departing from the spirit and scope of the embodiments in their broadest form.

What is claimed is:

1. An exercise form determination system for determining exercise form quality, the exercise form determination system comprising:
at least one sensor, said at least one sensor operating to capture raw data associated with an exercise movement;
an exercise form analysis module, said exercise form analysis module in communication with said at least one sensor such that said exercise form analysis module receives said raw data captured by said at least one sensor;
wherein said exercise form analysis module generates current actionable data based on said raw data and wherein said current actionable data is employed to make at least one exercise form determination based on said current actionable data.

2. The exercise form determination system of claim 1 wherein previously captured actionable data is paired with exercise form determination results to create a machine learning database.

3. The exercise form determination system of claim 2 wherein said current actionable data is processed in connection with data in said machine learning database to make at least one exercise form determination.

4. The exercise form determination system of claim 1 wherein said raw data is smoothed prior to the generation of actionable data based on said raw data.

5. The exercise form determination system of claim 1 wherein said sensor wirelessly communicates with said exercise form analysis module.

6. The exercise form determination module of claim 3 wherein at least one reference frame data element is employed in connection with said current actionable data to make at least one exercise form determination.

7. The exercise form determination module of claim 6 wherein said at least one reference frame data element comprises user height.

8. The exercise form determination module of claim 1 wherein said at least one sensor comprises an accelerometer.

9. The exercise form determination module of claim 1 wherein said at least one sensor comprises a gyroscopic sensor.

10. The exercise form determination module of claim 1 wherein said at least one sensor comprises a knee sleeve sensor operable to generate raw data associated with knee bend angles.

11. The exercise form determination module of claim 1 wherein said exercise form determination is reported to a user via a display.

12. A method for analyzing and determining the quality of exercise form comprising the steps of:
capturing raw data using one or more sensors;
smoothing said raw data to modify outlier data to generate smoothed raw data;
generating actionable data from said smoothed raw data;
employing said actionable data to generate lift quality determinations wherein said lift quality determinations comprise answers to questions associated with the form of a lifting exercise; and
reporting said answers on a display.

13. The method of claim 12 further comprising the step of receiving manual input data from a training user, said manual input data comprising said answers to questions associated with the form of a lifting exercise and wherein said manual input data is paired with said actionable data from a current lift and stored in a machine learning database.

14. The method of claim 13 wherein said machine learning database is employed in connection with future lift quality determinations associated with future lifting exercises.

15. The method of claim 12 wherein at least one reference frame data component is employed to generate said actionable data.

16. The method of claim 12 wherein said one or more sensors comprises at least one knee sleeve sensor.

17. The method of claim 12 wherein said one or more sensors comprises an accelerometer.

18. The method of claim 12 wherein said one or more sensors comprises a gyroscopic sensor.

19. The method of claim 12 wherein said lift quality determination comprises the question of whether a user’s knee buckled during a lifting exercise.

20. The method of claim 12 wherein said lift quality determination comprises the question of whether there was proper acceleration of a barbell during a lifting exercise.
